(19) United States
(12) Patent Application Publication
(10) Pub. No.: US 2006/0069305 A1
(43) Pub. Date: Mar. 30, 2006

(54) Device with enhanced indication of use and prevention of re-use

(75) Inventors: Lucien Alfred Couvillon JR., Concord, MA (US); Sheng-Ping Zhong, Shrewsbury, MA (US); Michael S. Banik, Bolton, MA (US); Ronald A. Sahatjian, Lenxington, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Boston Scientific Scimed, Inc.

(21) Appl. No.: 10/955,905

(22) Filed: Sep. 30, 2004

(51) Int. Cl. A61B 1/00; A61B 17/00

(52) U.S. Cl. 600/117; 606/1; 600/140

(57) ABSTRACT

In one aspect, the present invention is a medical device that reduces the likelihood of an operator improperly reusing it. The medical device includes (a) a device body; (b) a substrate comprising a contrast material that covers at least a portion of the device body; and (c) an outer layer comprising a hydrophilic material that covers at least a portion of the substrate. The outer layer comprising hydrophilic material is capable of absorbing water-soluble contaminants during clinical use that, once absorbed, are visible against the background of the substrate comprising the contrast material. In another aspect, the device includes (a) a device body and (b) an outer layer comprising a usage indicator reagent. In another aspect, the invention provides methods of preventing re-use of a single use medical device.

### FIELD OF THE INVENTION

The present invention relates to medical devices in general and prevention of re-use of medical devices in particular.

### BACKGROUND OF THE INVENTION

As an aid to the early detection of disease, it has become well established that there are major public health benefits from regular endoscopic examinations of internal structures such as the alimentary, excretory and reproductive canals and airways, e.g., the esophagus, lungs, colon, uterus, urethra, kidney and other organ systems. A conventional imaging endoscope used for such procedures comprises a flexible tube with a fiber optic light guide that directs illuminating light from an external light source to the distal tip where it exits the endoscope and illuminates the tissue to be examined. An objective lens and fiber optic imaging light guide communicating with a camera at the proximal end of the scope, or an imaging camera chip at the distal tip, produce an image that is displayed to the examiner.

Conventional endoscopes are expensive medical devices costing in the range of $25,000 for an endoscope, and much more for the associated operator console. Because of the expense, these endoscopes are built to withstand repeated disinfections and use upon many patients. Conventional endoscopes are generally built of sturdy materials, which decreases the flexibility of the scope and thus can decrease patient comfort. Furthermore, conventional endoscopes are complex and fragile instruments that frequently need expensive repair as a result of damage during use or during a disinfection procedure.

Single use disposable medical devices have become popular for instruments with small lumens and intricate, delicate working mechanisms that are difficult to sterilize or clean properly. Single use disposable devices packaged in sterile wrappers reduce the risk of pathogenic cross-contamination of diseases such as HIV, hepatitis, and other pathogens. Hospitals generally welcome the convenience of single use disposable products because of reduced concerns with product age, preparation and pre-test, overuse, breakage, malfunction and sterilization. However, with the advent of single use medical devices there are economic incentives among some users to attempt to re-use a single use medical device. Therefore, in order to prevent improper re-use, there is a need for medical devices and methods that ensure that medical devices intended for single use are in fact unused a second time.

### SUMMARY OF THE INVENTION

To address these and other problems in the prior art, the present invention is a medical device that reduces the likelihood of improper re-use. The medical device includes (a) a device body; (b) a substrate comprising a contrast material that covers at least a portion of the device body; and (c) an outer layer comprising a hydrophilic material that covers at least a portion of the substrate. The outer layer comprising hydrophilic material is capable of absorbing soluble contaminants during clinical use that, once absorbed, are visible against the background of the substrate comprising the contrast material. Therefore, the combination of the substrate comprising the contrast material and the outer layer comprising the hydrophilic material allows an observer to easily determine whether the medical device has previously been used by visually inspecting the outer surface of the device. In some embodiments, the hydrophilic material is covalently attached to the substrate. In some embodiments, the substrate further comprises a usage indicator reagent.

In another aspect, the invention provides a medical device that indicates prior use upon visual inspection, the medical device comprising a device body and an outer layer comprising a usage indicator reagent. The outer layer covers at least a portion of the device body. In some embodiments, the outer layer further comprises a hydrophilic material. In some embodiments, the usage indicator reagent is an organic dye molecule. In some embodiments the usage indicator reagent is a ferrous salt.

In another aspect, the present invention provides a method for reducing the likelihood of re-use of a medical device comprising a device body, a substrate comprising a contrast material covering at least a portion of the device body and an outer layer comprising a hydrophilic material covering at least a portion of the substrate. The method comprises visually inspecting the outer surface of a medical device prior to clinical use for the presence of absorbed contaminants, and disposing of or sterilizing a medical device that has absorbed contaminants.

In yet another aspect, the present invention provides a method for reducing the likelihood of re-use of a single use medical device comprising a device body and an outer layer comprising a usage indicator reagent covering at least a portion of the device body. The method comprises visually inspecting the outer surface of a single use medical device prior to clinical use for a change in color of a usage indicator reagent.

The medical devices and methods of the present invention can be used in any situation where there is a need to reduce the likelihood of improper clinical re-use of a medical device, such as a device intended for a single use in a living body, or the re-use of a device before sterilization.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagram illustrating a medical device in accordance with an embodiment of the present invention;

FIG. 2A shows a perspective view of a representative medical device of the invention with a portion of the outer layer comprising the hydrophilic material removed to expose the underlying substrate comprising the contrast material and the device body;

FIG. 2B shows a transverse cross-section of the medical device of FIG. 2A;

FIG. 3 illustrates a retractable sterile wrapper that covers at least a portion of a medical device in accordance with another embodiment of the present invention;

FIG. 4A shows a perspective view of a representative medical device of the invention with a portion of the outer layer comprising a usage indicator reagent removed to expose the underlying device body;

FIG. 4B shows a transverse cross-section of the medical device of FIG. 4A;

FIG. 5 is a flow diagram of a method for reducing the likelihood of improper re-use of a medical device with a contrast layer and an outer hydrophilic layer in accordance with an embodiment of the present invention; and

FIG. 6 is a flow diagram of a method for reducing the likelihood of improper re-use of a medical device with an outer layer comprising a usage indicator reagent in accordance with an embodiment of the present invention.

### DETAILED DESCRIPTION OF THE PREFERRED EMBODIMENT

Unless specifically defined herein, all terms used herein have the same meanings as would be understood by those of ordinary skill in the art of the present invention. The following definitions are provided to clarify the terms as they are used in the specification and claims to describe the present invention.

As used herein, the term “single use medical device,” and grammatical equivalents thereof, means a device that is intended for one use or use upon a single patient followed by disposal. The device may be completely or partially implanted into a living body, or otherwise exposed to a living body during the course of the normal operation of the device.

As used herein, the term “contaminant,” and grammatical equivalents thereof, refers to any material that contacts the single use device and renders it improper for further clinical use with that patient or upon another patient. Contaminants include, for example, bodily fluids, such as blood, excretory material such as urine or fecal fluids or solids, exfoliated cells, lymphatic material, exudates, mucosal secretions, and the like; which may contain proteins, metabolic products, bacterial flora, viruses, or endogenous compounds generated by normal or pathological processes in the body. Contaminants also include exogenous substances such as food products, medicines, drugs, nutritional supplements, vitamins and the like.

Generally described, the present invention provides medical devices that indicate prior clinical use. In some embodiments the devices of the invention may be used to reduce the likelihood of improper re-use through the use of a substrate comprising a contrast material that covers at least a portion of the device body and an outer layer comprising a hydrophilic material that covers at least a portion of the contrast substrate. Contaminants (bodily fluids, fecal material, sterilants, and the like) become absorbed in the hydrophilic coating material during clinical use and are visible against the contrast substrate. In other embodiments, the devices are at least partially covered with a layer comprising a usage indicator reagent. The usage indicator reagent undergoes a change in color or fluorescence upon a change in pH, and/or upon binding to protein, thus indicating prior clinical use. In some embodiments, the usage indicator reagent undergoes an irreversible change. In alternative embodiments, the usage indicator reagent undergoes a change that is reversible upon sterilization and/or treatment with an appropriate agent.

The medical devices of the invention may be used for any medical procedure that involves contact and/or insertion of the device within, or exposure to, a living body. In some embodiments, the medical devices are single use medical devices, such as endoscopes, imaging catheters, fiber optic guide wires and the like which are useful because they reduce or eliminate the need to sterilize and repair complex and fragile instruments that frequently need expensive repair as a result of damage during use or during a disinfection procedure. Medical devices of the invention may be adapted to be fully or partially inserted into a living body during the normal course of operation of the device. Representative examples of completely insertable devices include, but are not limited to, endoscopes, laparoscopes, vascular and nonvascular catheters, stents, and other devices and guidewires. Representative examples of partially insertable medical devices include biosensors and percutaneous access devices (such as introducers, access sheaths and catheters) that link the interior of a living body to the exterior of the body or an external medical device, such as a kidney dialysis machine. Some devices of the invention are adapted to be affixed to soft tissue of a living body such as the skin or mucous membranes. Examples of medical devices that are adapted to be affixed to soft tissue include wound or burn treatment devices.

The various embodiments of the devices and methods of the present invention may be applicable to any user who would benefit from devices and methods for reducing the likelihood of re-use of a medical device, such as, for example, manufacturers and retailers of medical devices, physicians, surgeons, nurses, paraprofessionals, and other medical personnel, as well as patients.

In one aspect, the present invention is a medical device that reduces the likelihood of improper re-use comprising (a) a device body; (b) a substrate comprising a contrast material that covers at least a portion of the device body; and (c) an outer layer comprising a hydrophilic material that covers at least a portion of the contrast substrate. In some embodiments, the medical device is a reusable device that requires sterilization after each use. In a preferred embodiment, the medical device is a single use device that is disposed of after contacting a living body.

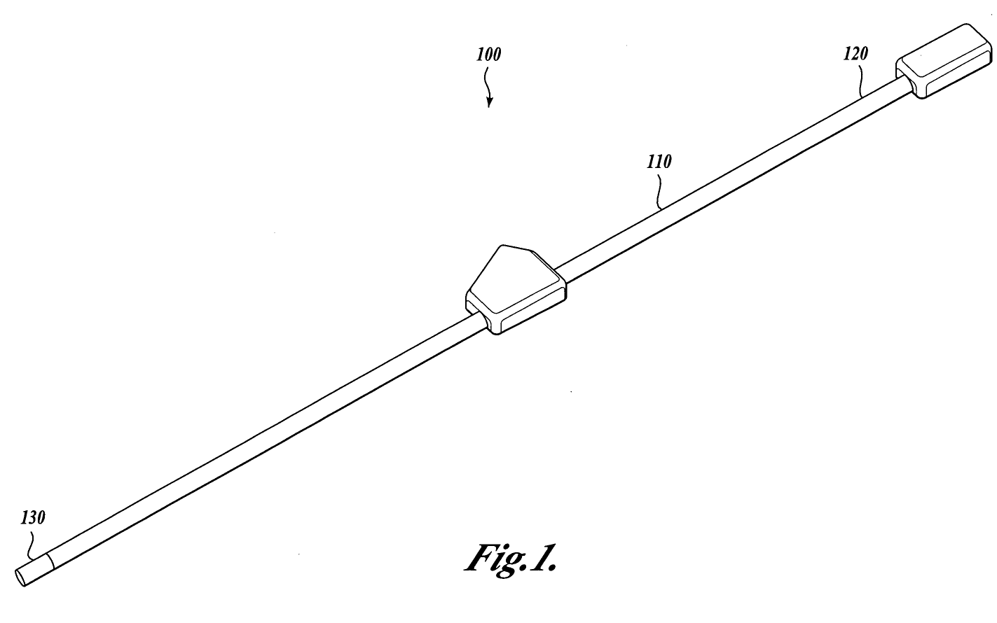

FIG. 1 illustrates one embodiment of a representative medical device in accordance with this aspect of the present invention. Medical device 100, shown here as an endoscope, comprises a device body 110 that contains one or more lumens for the purpose of performing endoscopic procedures and facilitating the insertion and extraction of fluids, gases and/or medical devices into and out of a living body. The device body 110 comprises a proximal end 120 attachable to a control unit for controlling the device functions and a distal end with a distal tip 130 comprising, in one example, an image sensor, optical lenses, a light source, such as a number of LEDs, and an articulating tip that enables steering of the device in a desired direction. Although the device illustrated in FIG. 1 is an endoscope, it will be understood by one of ordinary skill in the art that the device of the invention may be any medical device, not limited to endoscopes.

The device body 110 can be in any physical form (such as a tube, disk) and can be made from any suitable biocompatible material such as a polymeric or nonpolymeric substance or combinations thereof. Representative examples of biocompatible materials useful for making the device body include polypropylene, polyethylene, polyvinyl alcohol, polyvinylchloride (PVC), polyurethane (PU), polybutadienes (latex) polyester, polytetrafluoroethylene (PTFE), poly(lactic acid), poly(glycolic acid), polystyrene, polycarbonate, polyethylene glycol (PEG), fluoropolymers, collagen, poly(galactic acid), polyethylene terephthalate (PET), poly(dioxanone), poly(trimethylene carbonate) copolymers, poly (ε-caprolactone) homopolymers and copolymers, polyanhydrides, polyorthoesters, and copolymers of any of the foregoing. Other useful materials include silicone rubbers, polyamides, nylons, polycarbonates, polyaldehydes, natural rubbers, polyether-ester copolymers and styrene-butadiene copolymers. Examples of non-polymeric biocompatible materials include ceramics, metals, metal alloys, glasses and the like. Representative non-limiting examples of suitable metals include stainless steel or other biocompatible metal, titanium and NiTi superelastic or shape-memory materials. Combinations of polymeric substances and nonpolymeric substances as well as combinations of one or more polymeric substances and/or one or more on-polymeric substances can be used to form the device body. For example, representative composites useful for making the device body include, but are not limited to: hydroxyapatite and Bioglass®. In one embodiment, the device body is made of EG-80A durometer polyurethane.

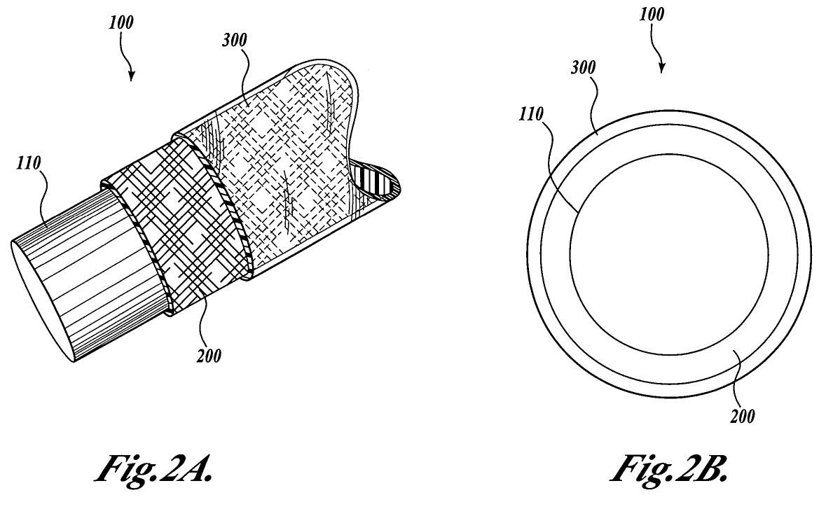

In accordance with this aspect of the invention, the device body 110 is at least partially covered with a substrate comprising a contrast material. FIG. 2A shows a perspective view of a representative medical device 100 with a portion of the outer layer removed to expose the underlying device body 110. As shown in FIG. 2A, the device body 110 is at least partially covered by a substrate 200 comprising the contrast material. The substrate 200 may cover either the whole of the device body 110, or may cover one or more portions of the device body, such as portions of the device intended to be inserted into a living body. The substrate 200 may be made of any suitable biocompatible contrast material, such as for example, a polymeric or metallic material. The contrast material is preferably white or light colored, but may be any material that provides a distinctive background upon which contaminants are capable of being visualized: i.e., white or light-colored on a dark background, or dark on a light background. For example, a white contrast substrate may be made by adding calcium carbonate filler to a polymeric material. In another example, a dark contrast material may be made by adding carbon black to a polymeric material. Additional, nonlimiting examples of suitable substrate materials include ceramic coatings such as titanium dioxide, zinc oxide, bismuth carbonate, barium sulphate or vacuum deposited material such as aluminum, or silver.

In some embodiments, the substrate 200 is chemically attached to a usage indicator reagent that changes color upon clinical use (as described in more detail below), which is in turn at least partially covered with the outer layer 300 comprising hydrophilic material. In such embodiments, the hydrophilic material may be chemically (e.g., covalently or ionically bonded) to the substrate and chemically attached to a usage indicator reagent. For example, if a chemically attached outer layer 300 becomes detached from the substrate (e.g., by mechanical abrasion) during clinical use, the usage indicator reagent will remain attached to the substrate 200 and its change in color will reveal prior use, as further described below. In another example, if a chemically attached outer layer material covers a substrate chemically attached to a usage indicator reagent, the presence of contaminants and/or a change in color indicate prior use.

The substrate 200 may be chemically attached to the device body 110 for example, by attachment of a polymeric composition such as an aqueous dispersion or emulsion of a polymer having organic acid functional groups and a polyfunctional crosslinking agent having functional groups being capable of reacting with organic groups, wherein the first polymeric layer is substantially cross-linked prior to the application of the second layer, as described in U.S. Pat. No. 5,702,754 and U.S. Pat. No. 6,048,620, incorporated herein by reference. Alternatively, in some embodiments, the surface of the device body 110 does not possess useful reactive groups, and the device body 110 can be treated with radio-frequency discharge plasma etching to generate reactive groups in order to allow deposition of the substrate 200 comprising the contrast material.

As shown in FIG. 2A, the substrate 200 is at least partially covered with an outer layer 300 comprising a hydrophilic material. FIG. 2B shows a transverse cross section of FIG. 2A. As shown in FIG. 2B, the outer layer 300 covers at least a portion of the substrate 200 which in turn covers at least a portion of the device body 110 of the medical device 100. The outer layer 300 can cover the whole of the device body 110, or one or more parts of the device body, such as portions of the device containing the substrate 200.

The outer layer 300 comprises a hydrophilic material that is biocompatible and capable of absorbing water-soluble bodily contaminants. In some embodiments of this aspect of the invention, the hydrophilic material is a lubricious coating such as a hydrophilic polymer or hydrogel. Hydrophilic polymers for use in lubricous layers are known in the art. The hydrophilic polymer may comprise monomer units from one or more monomers having organic acid functional groups, such as, for example, acrylic acid, methacrylic acid and isocrotonic acid. In addition, the hydrophilic polymer may contain monomer units from at least one hydrophilic monomer without any organic acid functional groups, such as vinylpyrrolidone and acrylamide. For example, the hydrophilic polymer may be acrylic acid-acrylamide copolymer (supplied by Allied Colloids as Versicol WN33). Yet another example of a hydrophilic material that is biocompatible is polyethylene oxide (PEO), also known as polyethylene glycol (PEG). In some embodiments, the layer 300 comprises the HYDROPASSTM hydrophilic coating available from Boston Scientific Corporation, of Natick, Mass., and described in U.S. Pat. Nos. 5,702,754 and 6,048,620, which are herein incorporated by reference.

In some embodiments, the layer 300 comprises a hydrogel coating that is composed largely of water molecules such as a polyurethane hydrogel coating. The layer 300 may additionally contain other hydrophilic polymers and other lubricious ingredients in addition to the hydrophilic polyurethane polymers.

The layer 300 comprising the hydrophilic material may be attached to the substrate 200 using any art-recognized method, such as through the reaction of organic functional groups on the hydrophilic polymers with a crosslinking agent contained in the substrate 200. As another example, the substrate 200 may be treated to allow covalent attachment of the layer 300. The layer 300 may be either coated onto the substrate 200 that is attached to the device body 110, or may be attached to the substrate 200 prior to the attachment of the substrate 200 to the device body 110.

The layer 300 comprising the hydrogel coating may be formed by plasma treating the polymeric plastic or rubber substrate 200 attached to the device body 110 or chemically treating the metal substrate 200 attached to the device body 110 to affix amine-containing groups onto the surface of substrate 200, applying a biocompatible hydrophilic polyurethane coating containing isocyanate groups which covalently bond to the amine containing groups, and exposing the covalently bonded polyurethane coating to water and thereby converting the layer 300 into a hydrogel.

Once the outer layer 300 is attached to the substrate 200 on the device body 110, the hydrophilic material in the layer 300 can be hydrated (i.e., activated for use) using any aqueous fluid. In some embodiments, the hydrophilic material is a hydrogel and upon hydration, the hydrogel has a water content of at least 70% of the weight of the hydrogel coating, such as at least 75%, such as at least 80%, such as at least 85%, such as at least 90%, such as at least 95%, up to in excess of 95% by weight, based upon the dry hydrophilic hydrogel polymers.

In operation of the device 100 formed in accordance with this aspect of the invention, the device contacts and/or is inserted into a living body and upon contact and/or insertion into the living body, the hydrophilic material in the outer layer 300 readily absorbs water-soluble contaminants such as the contents of the intestinal tract, bodily fluids, and sterilants. The substrate 200 provides a contrast background over which the contamination absorbed into the hydrophilic material is easily visualized. Upon removal of the device 100 from the living body, the water-soluble contaminants absorbed in the layer 300 cause a discoloration of the outer surface of the device 100 which is obvious to the physician and all others that view the used device, thus making improper re-use of the device 100 obvious to any person visualizing the used device such as medical personnel and non-medical personnel, including patients.

In some embodiments, the hydrophilic material in the outer layer 300 is hydrated and then is vacuum dried, packaged and sterilized before shipping the device 100. In such embodiments, the device 100 can be reactivated prior to clinical use by immersion into aqueous fluids. In other embodiments, the hydrophilic material in the layer 300 is hydrated and packaged prior to shipping.

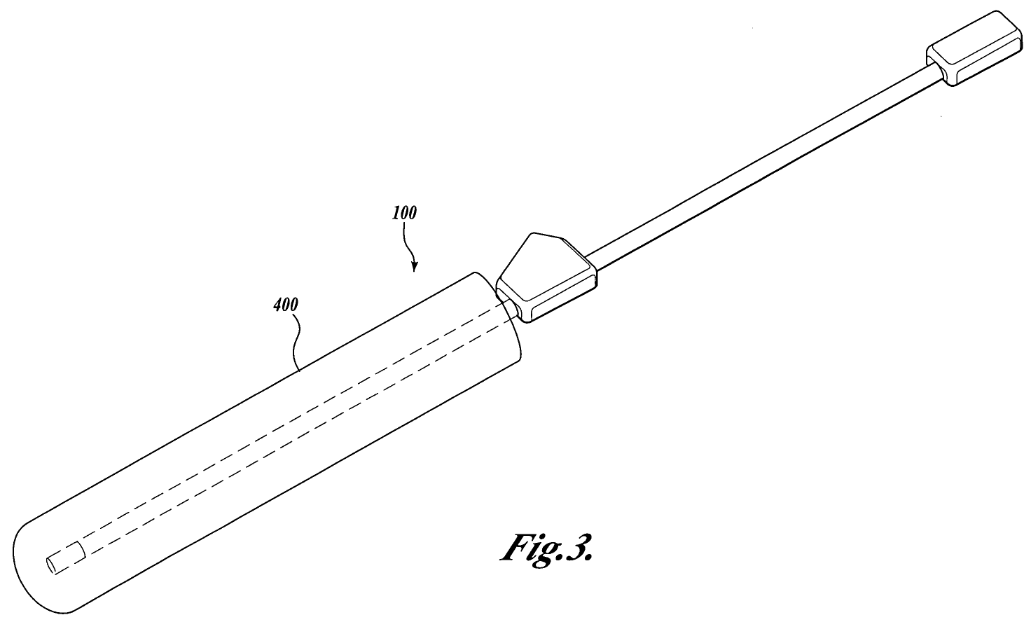

In numerous embodiments of the invention, as shown in FIG. 3, the medical device 100 further comprises a removable sterile wrapper 400 that operates to keep the device 100 clean and sterile and is removed prior to clinical use. In some embodiments, the hydrophilic material in the outer layer 300 of the device 100 is hydrated prior to packaging in the wrapper 400. In other embodiments, the device 100 is packaged in the wrapper 400 with a dried layer 300, which must be activated by hydration prior to clinical use.

The medical devices of the present invention having an outer layer 300 with a hydrophilic material have particular advantages over other devices that do not contain such layers. It has been recognized that hydrophilic materials offer good biocompatibility to medical devices when in contact with biological fluids or living tissue. In addition to biocompatibility, the hydrophilic outer layer is lubricous or “slippery” and reduces friction when the device is inserted into the human body, thereby allowing greater ease of manipulation and minimizing patient trauma. Moreover, the outer hydrophilic second layer in combination with the inner substrate comprising a visible contrast material allows the layer and substrate to function together as a usage indicator.

A particularly preferred embodiment of the medical device 100 according to the present invention is a single use imaging endoscope having at least the distal tip 130 provided with the outer layer 300. As an illustrative example, in one such embodiment, a prototype 10-13 mm diameter sheath having a 0.060 inner spiral wrap with a pitch of ¼ inch and coated with a layer 300 comprising a hydrophilic material was found to have a coefficient of friction of 0.15 compared to 0.85 for conventional endoscopes. As an additional example, an embodiment of an endoscope of the present invention only required 0.5 lbs. of force to push it through a 2-inch, U-shaped bend where a conventional endoscope could not pass through such a tight bend. Therefore, the present invention allows a single use endoscope to be made inexpensively and lightweight so that it is more comfortable for the patient due to a lower coefficient of friction and aids to prevent re-use, thereby reducing the risk of cross-contamination and enhancing patient safety.

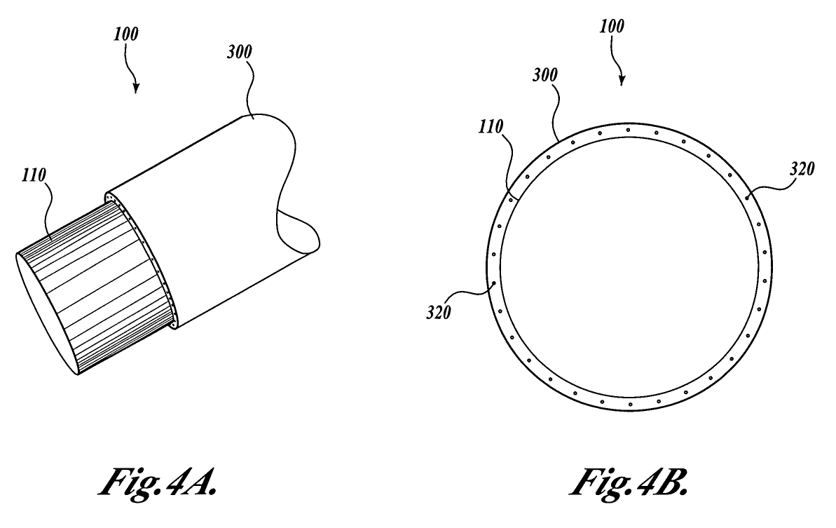

In another embodiment, the present invention provides a medical device that indicates prior use upon visual inspection comprising a device body at least partially covered with an outer layer comprising a usage indicator reagent. As shown in FIG. 4A, the device body 110 is at least partially covered with an outer layer 300. FIG. 4B shows a transverse cross-section of the medical device of FIG. 4A illustrating the device body 110 covered with an outer layer 300 comprising a usage indicator reagent 320. The outer layer 300 can cover the whole of the device body 110, or one or more portions of the device body 110.

The usage indicator reagent 320 may be any molecule that changes color in the visible spectrum or in the ultraviolet (UV) spectrum as a result of exposure to a living body. In some embodiments, the usage indicator reagent is an organic dye that changes color (either in the UV or visible spectrum) in the presence of an acidic environment (below pH 7.0). The device may contain an outer layer comprising an organic dye that is selected for use in a particular portion of the body. For example, the gastrointestinal tract is known to be very acidic, with the pH ranging from about 4.0 to about 1.0. Therefore, any organic dye molecule that changes color in the visible spectrum between a neutral pH of 7.0 and an acidic pH below pH 4.0 would be useful as a usage indicator reagent for a medical device used in the gastrointestinal tract. Several illustrative examples of organic dye compounds that change colors at an acidic pH value are shown in Table 1 below.

TABLE 1
pH Sensitive Organic Dyes

| | Color at | Color at | Color at | Color at |
|---|---|---|---|---|
| Organic Dye | pH 7.0 | pH 4.0 | pH 2.0 | pH 1.0 |
| Methyl violet | violet | violet | violet/green | green/yellow |
| Thymol blue | yellow | yellow | orange | red |
| Methyl yellow | Yellow | Orange | Red | red |
| Bromphenol blue | Blue | Green | Yellow | yellow |
| Methyl orange | Yellow | Orange | Red | red |
| Methyl red | Yellow | Red | Red | red |
| Litmus | Purple | Red | Red | red |
| Bromthymol blue | Blue | Yellow | Yellow | yellow |
| Phenol red | Orange | Yellow | Yellow | yellow |
| Phenolphthalein | Red | No color | No color | No color |

The colors listed above in TABLE 1 correspond to the visible wavelength ranges shown in TABLE 2 below.

TABLE 2
Wavelength Ranges of Dye Color

| | Wavelength Range in the Visible |
|---|---|
| Color | Spectrum |
| Violet | 400-420 nm |
| Indigo | 420-440 nm |
| Blue | 440-490 nm |
| Green | 490-570 nm |
| Yellow | 570-585 nm |
| Orange | 585-620 nm |
| Red | 620-780 nm |

In another embodiment, the usage indicator reagent is an organic dye molecule that changes color (either in the UV or visible spectrum) when complexed with protein or amino acid molecules. Proteins and amino acid molecules are present in body cavities and are therefore a useful indication of prior clinical use of a device. For example, the main constituents of intestinal juice are electrolytes, lipids and proteins, including albumin, gamma-globulins, mucoproteins and enzymes (see e.g. Riva et al., in Peeters, H. (Ed.) Protides of the Biological Fluids, Proceedings for the 11^{th} Colloquium, Brussels 1963, Elsevier, Amsterdam, 1964, p. 168).

Any dye which upon binding or reacting with a protein can undergo a change in spectroscopic properties can be used for protein determination and thereby indicate prior usage of the device. For example, azo dyes combine with the free basic amino groups of lysine, histidine and arginine and with the terminal amino groups of a protein chain. Examples of azo dyes include monoazo dyes (Orange G, Ponceau 2R), disazo dyes (Amido black 10B, Biebrich scarlet), trisazo dyes, and tetrakisazo dyes Isirius red F3B). The reactive dye Remazol Brilliant Blue R has been used as a protein indicator. Another example of a protein indicator dye is Coomassie Brilliant Blue G250 that forms a dye/protein complex formation commonly used to quantitate protein in a Bradford Assay (see Bradford U.S. Pat. No. 4,023,933). Additional illustrative examples of dyes that bind to proteins and undergo a change in spectroscopic properties are shown below in TABLE 3.

TABLE 3
Dyes that bind to protein

Protein Binding Dye
Acid Violet 17
Comassie Blue G250
Serva Blue W
Fluorescamine
o-phthaldialdehyde
Napthalene-2,3 ,-dicarboxaldehyde
NanoOrange ®
Ponceau S
Fast Green FCF
Eosin Y
Reactive Blue 15
Reactive Red 4
Stains-All
Benzyl Violet

Organic dye molecules may be covalently attached to the surface of the device body 110 to form the outer layer 300 by covalent activation of the surface of the device body 110. By way of representative example, organic dye molecules(s) can be attached to the device body by any of the following pairs of reactive groups (one member of the pair being present on the surface of the device body, and the other member of the pair being present on the matricellular protein(s): hydroxyl/carboxylic acid to yield an ester linkage; hydroxyl/anhydride to yield an ester linkage; hydroxyl/isocyanate to yield a urethane linkage. A surface of the device body 110 that does not possess useful reactive groups can be treated with radio-frequency discharge plasma etching to generate reactive groups in order to allow deposition of matricellular protein(s) (e.g., treatment with oxygen plasma to introduce oxygen-containing groups; treatment with propyl amino plasma to introduce amine groups).

Alternatively, the outer layer 300 may further comprise a hydrophilic material complexed with a usage indicator reagent 320 as previously described. In such embodiments, the outer layer 300 comprising a hydrophilic material such as a hydrogel can be attached as previously described for the attachment of the contrast substrate 200 to the outer layer 300. In some embodiments, the usage indicator reagent is an organic dye molecule that changes color in an environment with a pH below 7.0, or upon complexing with protein as previously described. As an illustrative example, a hydrophilic material may be complexed with NanoOrange® (available from Molecular Probes, Eugene, Oreg.), a usage indicator reagent which undergoes a dramatic fluorescence enhancement upon interaction with protein. Nanoorange® is capable of detecting protein in solution the range of 10 ng/mL to 10 ug/mL when fluorescence is measured at a wavelength of about 485/590 nm (Handbook of Fluorescent Probes and Research Chemicals, R. Haugland, 6^{th} Ed. Molecular Probes, Inc., p. 180-181 (1996)).

In operation, a device 100 containing a device body 110 comprising an outer layer 300 with hydrophilic material and a usage indicator reagent 320 such as an organic dye molecule contacts a living body. The hydrophilic material in the outer layer 300 absorbs liquid in an acidic aqueous environment such as the gastric tract and the organic dye molecule changes color within the hydrogel. This combination provides the advantage of having a lubricious coating as well as a usage indicator reagent. Prior use is indicated by absorption of contaminants and/or a change in color of the usage indicator reagent.

In another embodiment, the usage indicator reagent is a ferrous salt, such as FeSO₄ that is incorporated into an outer layer 300 comprising a hydrophilic material. When exposed to an aqueous environment, such as occurs during implantation into a living body, the hydrophilic material absorbs moisture and the ferrous salt marker oxidizes and changes color, thereby indicating prior use. In this embodiment, the ferrous salt marker can be added to the outer layer 300 comprising the hydrophilic material and stored in a dry form until clinical use.

Alternatively, the ferrous salt can be encapsulated in a protein layer prior to addition to the outer layer 300. In such embodiments, the hydrophilic material in the outer layer 300 can be hydrated and stored prior to clinical use, without triggering a color change because the encapsulated ferrous salt is not oxidized due to the protection of the protein layer. Clinical use is indicated upon exposure to enzymes present in a living body, such as such as pepsin in the gastric tract or pancreatin in the intestinal tract, wherein the protein encapsulating the ferrous salt is digested away and the ferrous salt oxidizes and changes color indicating prior use.

In another embodiment, the usage indicator reagent is an organic dye molecule (either fluorescent or visible spectrum dye) that is covalently linked to the device body 110 via a protein linkage. Upon exposure to a living body, the protein linkage is enzymatically cleaved (e.g. by enzymes in the gastric tract) from the device body 110, resulting in a visible reduction of dye on the surface of the device body 110.

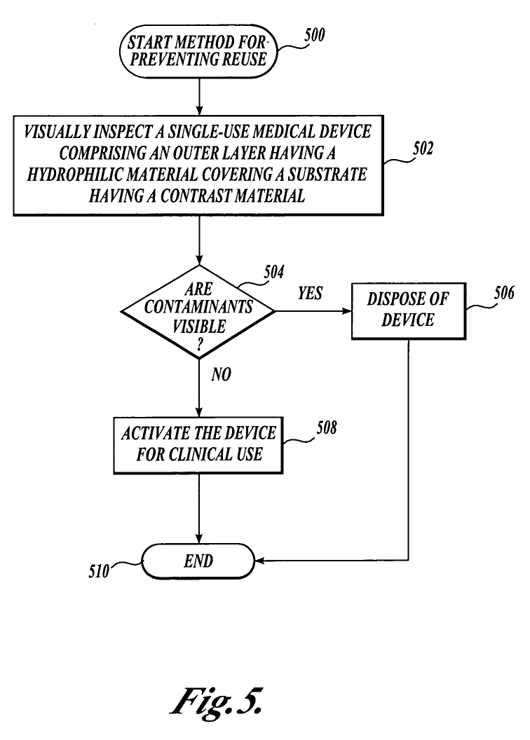

In another aspect, the present invention provides methods of reducing the likelihood of improper re-use of a medical device. The single use medical devices described herein are useful in the methods of this aspect of the invention. FIG. 5 is a flow chart of the method for reducing the likelihood of improper re-use of a medical device (e.g. a single use medical device) comprising a contrast substrate and an outer layer comprising hydrophilic material. The method begins at 500 and comprises visually inspecting a single use device for the presence of absorbed contaminants at 502, wherein the single use device comprises a device body, a substrate comprising a contrast material that covers at least a portion of the device body and an outer layer comprising a hydrophilic material that covers at least a portion of the substrate. A visual inspection test is made at 504 to determine if absorbed contaminants are observed in the outer layer of the device. The visual inspection test can be made by a physician or other medical personnel, or by an imaging device. If contaminants are detected (i.e., coloration such as red or brown staining of the outer layer), the device is disposed of at 506. If no contaminants are detected, activation of the device is allowed at 508 and the method terminates at 510.

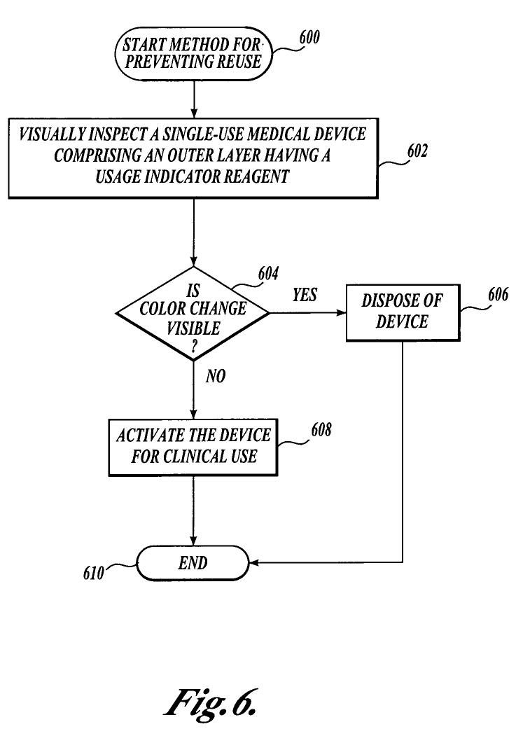

FIG. 6 is a flow chart of the method for reducing the likelihood of re-use of a single use medical device comprising an outer layer comprising a usage indicator reagent. The method begins at 600 and comprises visually inspecting a single use device for a color change in a usage indicator reagent at 602, wherein the single use device comprises a device body and an outer layer comprising a usage indicator reagent that covers at least a portion of the device body. A visual inspection test is made at 604 to determine if a color change is observed in the outer layer of the device. The visual inspection test can be made by a physician or other medical personnel, or by an imaging device.

While the preferred embodiment of the invention has been illustrated and described, it will be appreciated that various changes can be made therein without departing from the scope of the invention. It is therefore intended that the scope of the invention be determined from the following claims and equivalents thereof.

The embodiments of the invention in which an exclusive property or privilege is claimed are defined as follows:

1. A medical device that indicates prior use upon visual inspection, the medical device comprising:
(a) a device body;
(b) a substrate comprising a contrast material that covers at least a portion of the device body; and
(c) an outer layer comprising a hydrophilic material that covers at least a portion of the substrate;
wherein the hydrophilic material absorbs water-soluble contaminants during clinical use of the medical device; and wherein the absorbed contaminants are visible against the contrast material.

2. The medical device of claim 1, wherein the device body comprises a polymeric material.

3. The medical device of claim 1, wherein the contrast material is white.

4. The medical device of claim 1, wherein the hydrophilic material is covalently attached to the substrate.

5. The medical device of claim 1, wherein a usage indicator reagent is covalently attached to the substrate.

6. The medical device of claim 1, wherein the hydrophilic material is capable of becoming lubricous when contacted with an aqueous medium.

7. The medical device of claim 6, wherein the hydrophilic material is an acrylic acid-acrylamide copolymer.

8. The medical device of claim 6, wherein the hydrophilic material is a hydrogel.

9. The medical device of claim 8, wherein the hydrogel is capable of having a water content of at least 70%.

10. The medical device of claim 1, further comprising a sterile wrapper that is removed prior to use.

11. The medical device of claim 1, wherein the device is a single use endoscope.

12. The single use medical device of claim 1, wherein the hydrophilic material covers at least a distal tip of the endoscope.

13. A method for reducing the likelihood of improper re-use of a single use medical device comprising:
visually inspecting the outer surface of a single use medical device for the presence of absorbed contaminants, wherein the single use medical device includes:
(a) a device body;
(b) a substrate comprising a contrast material that covers at least a portion of the device body; and
(c) an outer layer comprising a hydrophilic material that covers at least a portion of the substrate; and
disposing of a single use medical device that contains absorbed contaminants in the outer layer of the device.

14. The method of claim 13 wherein the single use medical device is an endoscope.

15. The method of claim 13 wherein the hydrophilic material is a hydrogel.

16. A medical device that indicates prior use upon visual inspection, the medical device comprising:
(a) a device body; and
(b) an outer layer comprising a usage indicator reagent, wherein the outer layer covers at least a portion of the device body.

17. The medical device of claim 16 wherein the outer layer further comprises a hydrophilic material.

18. The medical device of claim 16 or 17 wherein the usage indicator reagent is an organic dye molecule.

19. The medical device of claim 18 wherein the organic dye molecule changes color in an environment with a pH below pH 7.0.

20. The medical device of claim 18 wherein the organic dye molecule changes color when complexed with protein.

21. The medical device of claim 16 wherein the usage indicator reagent is a ferrous salt.

22. The medical device of claim 21 wherein the ferrous salt is encapsulated in protein.
